(19) United States
(12) Patent Application Publication
(10) Pub. No.: US 2007/0173702 A1
(43) Pub. Date: Jul. 26, 2007

(54) Automatic Patient Healthcare and Treatment Outcome Monitoring System

(75) Inventors: Mary Dlugos, King of Prussia, PA (US); Jean Grigsby, Downingtown, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS HEALTH SERVICES CORPORATION, Malvern, PA (US)

(21) Appl. No.: 11/468,012

(22) Filed: Aug. 29, 2006

Related U.S. Application Data
(60) Provisional application No. 60/729,062, filed on Oct. 21, 2005.

(51) Int. Cl. A61B 5/00

(52) U.S. Cl. 600/300; 128/923

(57) ABSTRACT

A system generates criteria associating subjective narrative text describing a treatment outcome with acquired patient medical data and linking a narrative treatment outcome description to documented data during patient assessment documentation. A patient healthcare monitoring system includes an acquisition processor for automatically acquiring data representing outcomes of multiple corresponding assessments of medical condition of a patient. A data processor automatically evaluates a logical expression comprising a logical combination of the multiple corresponding assessments to provide an overall outcome representative result and compares the overall outcome representative result with a predetermined treatment objective of the patient to provide a result indicative of whether the treatment objective is met. A documentation processor updates a record of the patient to indicate the objective is met.

This is a non-provisional application of provisional application Ser. No. 60/729,062 by Mary Dlugos et al filed Oct. 21, 2005.

### FIELD OF THE INVENTION

This invention concerns a patient healthcare monitoring system involving automatically evaluating patient assessment information for comparison with treatment objectives.

### BACKGROUND OF THE INVENTION

In existing systems, a current workflow (task sequence including automated or manual tasks) requires a healthcare worker to document patient observations and Findings while providing care to the patient and to review the documentation documented in order to evaluate whether the outcomes for the patient have been met as measured with previously selected standard goals. A progress assessment is either written on paper or entered into a patient electronic care plan. This redundant documentation is time consuming for a healthcare worker whose main focus is providing care to patients. In order to meet requirements set by regulatory agents, such as the Joint Commission on Accreditation of Healthcare Organizations (JCAHO) and healthcare payer organizations, Healthcare Organizations have identified best practices and implemented standard guidelines for the care of specific patient problems and procedures. Frequently formal care plans reflect these best practices and can serve as a means for tracking whether defined best practices are being followed, and whether they are proving to be effective in the treatment of the patients to which they are applied. Evidence for the efficacy of care is often determined by whether a patient meets the expected goals of the plan and ultimately that problems are either resolved or stabilized

Treatment Goals and expected-outcomes are measurable clinical objectives that are established for a patient in relation to an identified problem. They are expected to be achieved as a result of implementing interventions stated in a plan of care. Target timeframes are assigned to the goals to be sure that patient condition improves at an accepted rate, using commonly accepted standards. A health care provider needs to document whether goals are met within timeframes indicated within the plan. Nurses and other healthcare workers, such as case managers, dietitians, respiratory therapists, physical therapists and physicians, are the typical personnel that manage care plans. These healthcare workers are often responsible for performing services contained within a care plan. Monitoring expected treatment outcomes requires constant vigilance and documentation by healthcare workers involved in the care plan. In a health care environment of decreased staff to patient ratios, the expectation that this documentation be completed in a timely manner is sometimes burdensome and unrealistic.

In existing systems, treatment outcome documentation is vulnerable to subjective evaluation if outcomes are not consistently quantified. In response to carrying out interventions indicated in a care plan a healthcare worker documents Findings. For example, a worker documents patient respiratory rate, documents the degree of edema, weighs the patient, documents the breath sounds, and looks at an X-Ray result. Also in existing systems (manual or automated) a worker accesses a care plan and indicates the status of the outcome, reads the outcome again and decides if the patient has achieved the desired results and then document status as ‘met’, ‘not met’, ‘progressing’, or with similar terminology. The interpretation of a status may be subjective and depend on the healthcare worker involved. A system according to invention principles addresses these deficiencies and related problems.

### SUMMARY OF THE INVENTION

A system generates criteria associating subjective narrative text describing a treatment outcome with acquired patient medical data during patient assessment documentation in realtime and provides a consistent standard evaluation of a treatment outcome without requiring programming. A patient healthcare monitoring system includes an acquisition processor for automatically acquiring data representing outcomes of multiple corresponding assessments of medical condition of a patient. A data processor automatically evaluates a logical expression comprising a logical combination of the outcomes of multiple corresponding assessments to provide an overall outcome representative result and compares the overall outcome representative result with a predetermined treatment objective of the patient to provide a result indicative of whether the treatment objective is met. A documentation processor updates a record of the patient to indicate the objective is met.

### BRIEF DESCRIPTION OF THE DRAWING

FIG. 1 shows a hospital information system including a patient healthcare monitoring system for use in patient assessment, according to invention principles.

FIG. 2 shows a flowchart of a process used by a patient healthcare monitoring system in generating criteria for associating subjective narrative text describing a treatment outcome with acquired patient medical data, according to invention principles.

FIG. 3 shows a flowchart of a process employing a patient healthcare monitoring system in making a patient assessment, according to invention principles.

FIG. 4 shows a displayed treatment outcome assessment associated with text describing a treatment outcome, according to invention principles.

FIG. 5 shows a displayed menu indicating a logical assessment expression, according to invention principles.

FIG. 6 shows a table indicating medical problems and associated treatment outcomes, according to invention principles.

FIG. 7 shows a flowchart of a process used by a patient healthcare monitoring system, according to invention principles.

### DETAILED DESCRIPTION OF THE INVENTION

A system according to invention principles improves documentation (e.g., of a patient medical condition) by using a standard catalog of terms and providing consistency and uniformity of documentation accessed by multiple healthcare workers. Descriptions of treatment outcomes may be subjective and indefinite if not consistently quantified, ideally in the same way. The system provides descriptions of treatment outcomes that are definite, concrete and measurable and automatically manages analysis and documentation to ensure compliance and standardization of healthcare documentation employed by healthcare workers in the various departments and specialties that provide care. The system assignment of standardized treatment objectives or goals for a patient facilitates measurement of quantitative outcomes and quality control in the treatment of multiple patients. The system employs evidence-based medicine and objective criteria in measuring patient progress and outcomes in meeting treatment objectives.

Treatment objectives and expected outcomes are quantifiable or measurable clinical objectives that are established for a patient concerning one or more identified medical problems. Treatment objectives are intended to be achieved through implementation of interventions indicated in a patient plan of care. For example, an ‘Ineffective breathing pattern’ is a problem that would have an expected outcome of ‘Patient maintains effective breathing pattern, as evidenced by respiratory rate 12 to 20 breaths/min with clear and equal lung sounds bilaterally and a chest x-ray shows lungs are fully expanded. Further, target timeframes may be assigned to treatment objectives in a care plan to ensure that a patient condition improves at an acceptable rate. A healthcare provider (HP) needs to document whether the goals are met within timeframes indicated within a care plan. The measurement of patient condition to determine if a treatment objective is met is usually based on the results (Findings) of a patient assessment, diagnostic studies, or clinical observations. The measurable goal can be a single Finding or a composite of multiple Findings typically documented by a healthcare worker providing care to the patient.

A healthcare worker performing interventions for a patient, makes and documents observations for an assessment. For example, the worker documents the respiratory rate, documents the degree of edema, weighs a patient, documents breath sounds, and reviews an X-Ray result. Upon completion of a patient physical examination, a healthcare worker returns to a nursing station to document the examination, using the care plan to indicate the status of a treatment outcome. In existing known systems, a healthcare worker reads a description of a treatment outcome again and determines if the patient has achieved the desired results and documents patient status indicating a treatment objective as being met, not met, progressing, or similar terminology.

In contrast, the system enables a non-programmer to construct criteria to associate a description of a treatment outcome with patient medical data thereby enabling a healthcare worker to document “real time” treatment outcomes using standardized, uniform data employed by multiple different healthcare workers. The system obviates the need for creation of code or an extra module, or the need for healthcare worker training in use of a rules engine or detailed technical SQL programming knowledge. The system also eliminates a need for specialists to create or update assessment rules and values, and the system measures the assessments objectively using tables of previously defined values that are consistently applied irrespective of the healthcare worker. The system also eliminates duplicate documentation while documenting patient outcomes.

An executable application, as used herein, comprises code or machine readable instruction for implementing predetermined functions including those of an operating system, healthcare information system or other information processing system, for example, in response to a user command or input. An executable procedure is a segment of code (machine readable instruction), sub-routine, or other distinct section of code or portion of an executable application for performing one or more particular processes and may include performing operations on received input parameters (or in response to received input parameters) and provide resulting output parameters. A processor as used herein is a device and/or set of machine-readable instructions for performing tasks. A processor comprises any one or combination of, hardware, firmware, and/or software. A processor acts upon information by manipulating, analyzing, modifying, converting or transmitting information for use by an executable procedure or an information device, and/or by routing the information to an output device. A processor may use or comprise the capabilities of a controller or microprocessor, for example. A display processor or generator is a known element comprising electronic circuitry or software or a combination of both for generating display images or portions thereof. A user interface comprises one or more display images enabling user interaction with a processor or other device.

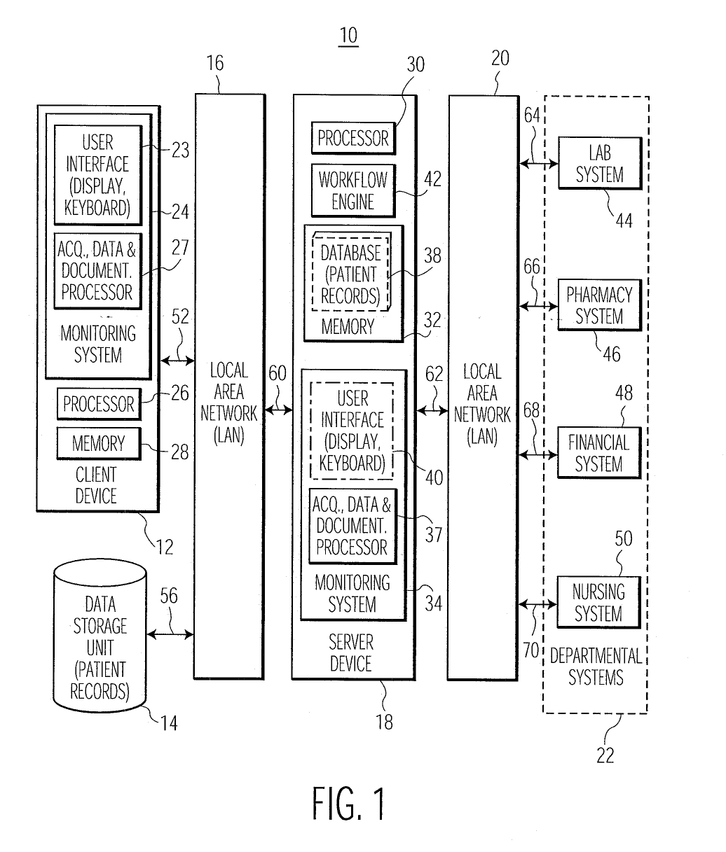

FIG. 1 shows a networked Hospital Information System (HIS) 10 including a patient healthcare monitoring system 34 for use in patient assessment. Healthcare monitoring system 34 provides criteria associating subjective narrative text describing a treatment outcome with acquired patient medical data during patient assessment documentation in real-time and provides a consistent standard evaluation of a patient treatment outcome. Healthcare monitoring system 34 may alternatively be located in client device 12 as unit 24 (or elsewhere in a network employed by system 10). Healthcare monitoring system 34 includes an acquisition processor, a data processor and a documentation processor (unit 37) and user interface 40. Client device 12, preferably implemented as a personal computer, also includes a processor 26, and a memory unit 28. Processor 26 and memory unit 28 are constructed and operate in a manner well known to those skilled in the art of the design of client devices.

Healthcare monitoring system 24 in client device 12 includes a corresponding acquisition processor, data processor and documentation processor (unit 27) and user interface 23. Healthcare information system 10 generally includes a client device 12, a data storage unit 14, a first local area network (LAN) 16, a server device 18, a second local area network (LAN) 20, and departmental systems 22. The healthcare information system 10 is used by a healthcare provider that is responsible for monitoring the health and/or welfare of people in its care. Examples of healthcare providers include, without limitation, a hospital, a nursing home, an assisted living care arrangement, a home health care arrangement, a hospice arrangement, a critical care arrangement, a health care clinic, a physical therapy clinic, a chiropractic clinic, and a dental office. In the preferred embodiment of the present invention, the healthcare provider is a hospital. Examples of the people being serviced by the healthcare provider include, without limitation, a patient, a resident, and a client.

User interface 40 in system 34 generally includes an input device that permits a user to input information and an output device that permits a user to receive information. Preferably, the input device is a keyboard and mouse, but also may be a touch screen or a microphone with a voice recognition program, for example. The output device is a display, but also may be a speaker, for example. The output device provides information to the user responsive to the input device and receives information from the user or responsive to other activity in client device 12. For example, the display presents information responsive to the user entering information via the keyboard.

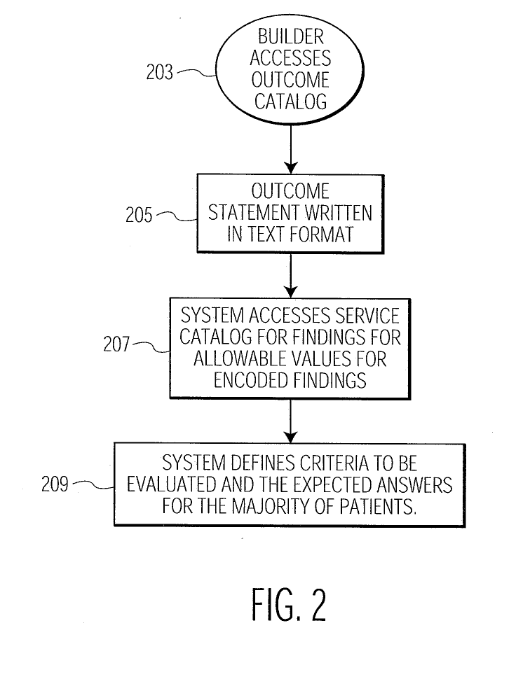

FIG. 2 shows a flowchart of a process used by patient healthcare monitoring system 34 in generating criteria for associating subjective narrative text describing a treatment outcome with acquired patient medical data. Criteria used to evaluate treatment outcomes are determined by a treatment outcome process that determines criteria used to calculate treatment outcome status of a patient using a catalog including predetermined outcome descriptions comprising text describing outcomes. Catalogs are used in HIS 10 for determining various system elements. For example, an order for a treatment (e.g., a record of a request for provision of a medication or procedure) that may be initiated by a physician is specified in a Service catalog as services with associated attributes. A problem associated with a patient is specified in a problem catalog and a treatment outcome associated with a patient is specified in a treatment outcome catalog, etc. Patient medical result data to be entered manually or received via an interface is specified in the Service catalog as Findings. In the Service catalog, attributes indicating whether a patient medical result is numeric, encoded (e.g., a patient medical parameter value), or text are specified. Numeric Findings are further specified in the Services catalog to indicate maximum high and low values as well as normal, abnormal, and critical ranges based on patient age and/or gender.

HIS 10 includes an Adaptability unit in healthcare monitoring system 34 providing user selectable option values comprising allowable values for a patient medical result data element corresponding to a defined Finding. System 34 also supports manual data entry of encoded data comprising a patient medical data value. For example, a respiration Finding is specified to be numeric together with various ranges indicating a child respiratory rate is expected to be higher than that of an adult. A Right Lower Lung Lobe Breath Sounds Finding associated with acquisition of data regarding breath sounds is designated as encoded in Findings data in a Service catalog. A corresponding data element in an Adaptability unit in system 34 employs a selection list of allowable encoded values including, Clear, Decreased, Crackles, Wheezes, Rhonchi, and Absent. A clinician selects an item from the selection list of allowable values in documenting a patient condition assessment. Individual acquired patient and other data items are collated and stored by system 34 in HIS 10. Healthcare monitoring system 34 acquires catalogs and Adaptability unit data elements within HIS 10, collates them together in a way that allows for treatment outcome criteria evaluation and determines if a result of the evaluation equals a logical TRUE value. If so, system 34 indicates a treatment outcome evaluation is true and a message is communicated to a clinician (e.g., via a display image, email, voice message etc.) that the treatment outcome has met predetermined outcome criteria.

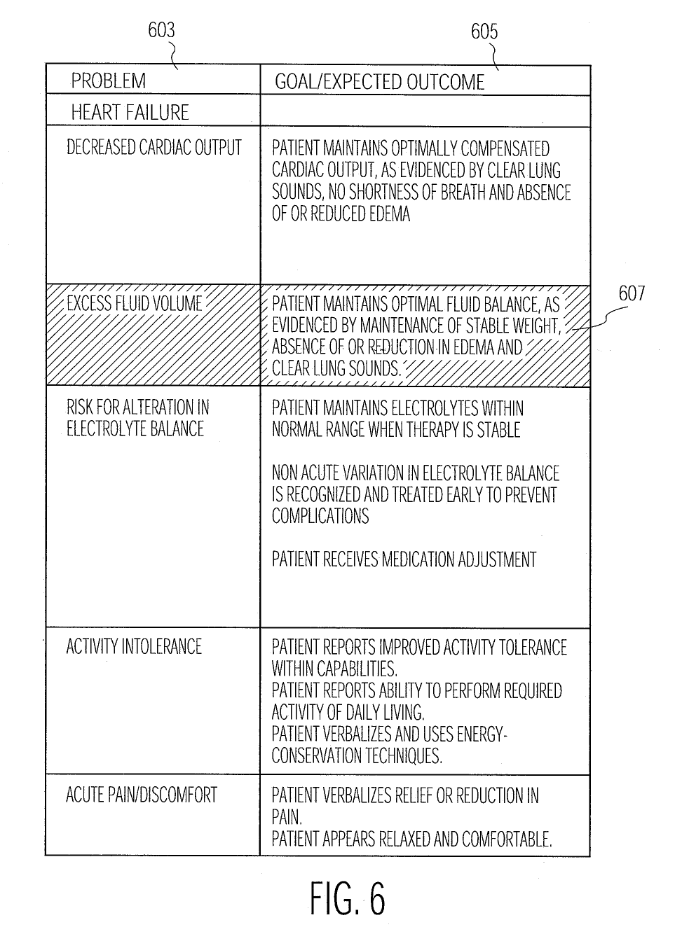

In step 203 a treatment outcome criteria generator in system 34 accesses an Outcome Catalog in response to a user or received command identifying an Outcome and in step 205 displays, via user interface 40, an associated treatment outcome description in text format derived from the Outcome catalog. FIG. 6 shows a table indicating medical problems and associated treatment outcomes associated with a diagnosis of Heart Failure. The problems and outcomes defined by standards groups such as the North American Nursing Diagnosis Association (NANDA) and Nursing Outcomes Classification (NOC) use common nursing nomenclature taxonomy. The table illustrates the difficulty experienced by healthcare workers in documenting treatment outcomes. Column 603 indicates a medical problem associated with Heart Failure and column 605 indicates a corresponding expected text treatment outcome description. The determination of whether a treatment outcome has been met and patient treatment is progressing to plan is a cumbersome and error-prone process in existing systems and typically involves the acquisition and review of multiple patient parameter values and data by healthcare workers.

An exemplary problem of excess fluid volume illustrated in row 607 has a corresponding treatment outcome of maintaining optimal fluid balance as evidenced by maintenance of stable weight and absence of, or reduction in, edema together with clear lung sounds. System 34 monitors acquired patient Findings including monitoring a patient medical data for a change of greater than two pounds weight in one day or a trend of predetermined weight gain over several days. Further, if a patient treatment involves restricting fluid intake, system 34 reviews patient data indicating recorded fluid intake and evaluates urine output in response to diuretics, to determine if intake or output exceeds predetermined thresholds. Similarly, system 34 monitors patient medical data to identify potential side effects of diuretics including hypolkalemia, hyponatremia, hypomagnesia, elevated serum creatinine level, and hyperuricemia. System 34 further monitors Findings derived from clinical assessments including, an assessment of the presence of edema by palpating a patient anatomical area over tibia, ankles, feet, and sacrum, ausculating lung sounds and assessing for labored breathing and jugular vein distention and ascites and monitoring abdominal girth

System 34 in conjunction with workflow engine 42 initiates activities in response to evaluation of a logical expression based on the previously determined excess fluid volume Findings and determination a treatment outcome has been achieved, or is improving, or is not improving. System 34 assesses treatment outcomes including for example, maintenance of stable weight in response to a Value of Weight Finding compared to a previously recorded Weight. An expression determines a stable weight TRUE result if a latest weight value is less than or equal to a previous weight value over a duration of 3 days. System 34 determines absence of, or reduction in edema as a treatment outcome, in response to a Finding CVE (Cardio-Vascular Edema) Edema Noted, for example. If a documented Finding answer is no, the condition is TRUE and if a documented answer is yes, system 34 evaluates the Finding of Grade for each of the locations (ankle, foot, calf, knee, hand, general, sacral). If a latest value of Grade is less than previously acquired values of Grade over 3 days the condition of absence or reduction in edema is TRUE. System 34 determines a clear lung sound treatment outcome in response to Findings of RUL (Right Upper Lung) Breath Sounds, RML (Right Middle Lung) Breath Sounds, RLL (Right Lower Lung) Breath Sounds, LUL (Left Upper Lung) Breath Sounds and LLL (Left Lower Lung) Breath Sounds. If a documented answer for these Findings indicates Clear, a logical expression is resolved by system 34 to indicate a clear lungs TRUE value. System 34 determines an Optimal fluid balance overall treatment outcome logical expression value in response to determining that the above conditions (stable weight, edema, clear lungs) are TRUE and a calculation of daily fluid balance based on recorded Input and Output assessment. Specifically, the calculation of daily fluid balance is determined to be TRUE if daily patient fluid output is greater than or equal to daily fluid input for a duration of 3 days. System 34 initiates generation of a message to a user indicating a “Patient Maintains optimal fluid balance” in response to an Optimal fluid balance overall treatment outcome logical expression value being TRUE (indicating a status of outcome MET).

System 34 provides a Health care entity with an ability to determine when a treatment outcome logical expression evaluation and user notification occurs. A configuration function in system 34 enables a user to configure logical expression evaluation or user notification to occur in response to, data representing a patient medical assessment being obtained or saved, a patient care plan being opened, user logon to a patient assessment, care plan or administration system or a work list indicating tasks to be performed by a worker indicates treatment outcomes are overdue for a patient or a user command. System 34 assesses patients who are assigned to a user and for which the user is authorized to access data.

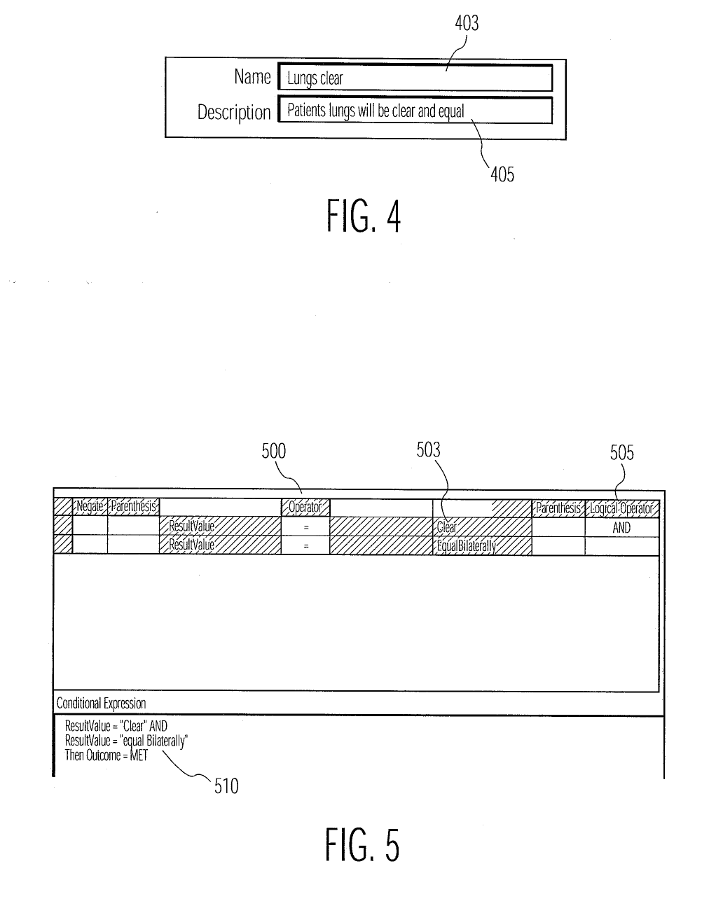

FIG. 4 illustrates a displayed treatment outcome assessment (Lungs Clear 403) associated with text describing a treatment outcome (Patient's lungs will be clear and equal 405). System 34 parses the treatment outcome description to identify one or more key words and phrases of the Outcome description associated with one or more corresponding Findings in a Service catalog and retrieves corresponding Findings information in step 207. The Findings include encoded Findings comprising logical expressions that may be calculated and evaluated by system 34 in response to user entered or automatically acquired patient parameter data values, data value ranges, quantitative (e.g., numbers, characters, specific data) values and qualitative (e.g., visual or auditory user entered assessment indicators, including, sounds, colors, shades, textures, thicknesses, patterns etc.). System 34 in step 209 automatically (and/or manually in another embodiment in response to user command) determines criteria comprising a logical expression to be evaluated to determine whether a treatment outcome has been met for treatment of a patient medical condition.

FIG. 5 shows a displayed menu 500 indicating logical assessment expression 510 automatically generated in response to accessing a treatment outcome description in text format in an Outcome catalog. Logical assessment expression 510 indicates a treatment Outcome is met if Lungs are Clear AND Equal Bilaterally. The expression Findings and operators are indicated in columns 503 and 505 respectively enabling a user to alter the automatically generated expression. A Finding employed in a generated logical expression may involve an expected patient parameter result. System 34 determines whether expected results have been obtained for a particular patient based on historical treatment outcome data for the treatment concerned. The expected outcome results arc determined based on treatment outcomes experienced by the largest proportion of patients of a patient population associated with the historical treatment outcome data for the treatment concerned.

System 34 in step 209 automatically identifies Findings that are associated with a treatment outcome description in text format derived from an Outcome catalog. The Findings are used in evaluation of a logical expression evaluated to determine whether a treatment outcome has been met and to identify values comprising a logical ‘true’ (e.g., a logical 1) of one or more terms or arithmetical products comprising the logical expression as well as to identify a value of the overall logical expression. A logical expression generation function in system 34 supports logical operators including, = (equals), < (less than), > (greater than), ‘contains’, ‘< >’ (less than or greater than), AND, OR, NOT operands and other operands to construct a logical expression. System 34 automatically selects Findings that are associated with a treatment outcome description from a list of Findings in a Service catalog using a search and look up function and/or predetermined mapping information associating treatment outcome types with corresponding Findings.

In one embodiment in response to selection of a Finding, system 34 initiates generation of a prompt message via a displayed menu provided by user interface 40 prompting a user to provide necessary criteria. For example, if system 34 selects a Respirations Finding, a user is prompted to provide a numeric value for use in a generated logical expression. If a Right Lower Lung Lobe Breath Sounds Finding is selected, system 34 prompts a user to select from a list of allowable values for this Finding provided by an Adaptability unit in system 34 during generation of a logical expression. If a selected Finding is defined as text or as not being restricted to associated values provided by the Adaptability unit, a user is able to enter a text phrase to be looked for during the evaluation. For example, in order to evaluate a Chest X-Ray result, a user enters ‘lungs fully expanded’ with an operand of ‘contains’. System 34 generates a logical expression from selectable items which advantageously simplifies the generation process and ensures that a logical expression correlates with medical data being collected from a patient. In another embodiment in response to selection of a Finding, system 34 automatically provides necessary criteria (including text and values) associated with a Finding using predetermined mapping information associating criteria with corresponding Findings.

The logical expression generation function in system 34 combines Findings using AND and OR operands, to provide expressions such as if Finding A OR Finding B is True, or if Finding A=x AND Finding B=y, or if Finding A is >20 AND <32. Other exemplary generated logical expressions types include, for example, make sure A is blank or if Finding A is Not Valued perform action x. Further, logical expressions employ a ‘contains’ operator, e.g., an X-Ray result contains the phrase ‘lungs fully expanded’ If Chest X-Ray contains ‘lungs fully expanded’. Expressions may also include an option to compare previously acquired patient medical parameter values for differences, e.g., first pain scale is 7 so if it is reduced by 3 then=True. An expression or Finding in an expression may also require a time range or number of values for a True result, e.g., 80 percent of measurements of patient respiratory rate are needed to be between 12 and 20 over a 2 day period for a True result.

The logical expression generation function in system 34 enables a non-programmer with minimal information technology skills, to generate a logical expression linking a narrative treatment outcome description to acquired patient medical parameter data. The logical expression generation function enables a user to define triggers which use Findings derived from clinician assessments, interventions and results in order to conditionally prompt the user that treatment Outcome Measurements associated with a Patient Problem have been met, partially met or complete, depending on the established trigger conditions. System 34 automatically links a specified treatment outcome to acquired patient medical Findings in response to events such as upon incorporation of an Outcome definition description in an outcome catalog, for example. A generated logical expression associated with a narrative treatment outcome description employs information derived from other definitional catalogs and tables in initiating comparisons and other operations involving acquired patient medical parameter data. A logical expression is employed to automatically determine when acquired patient medical parameter data complies with a treatment Outcome description. A logical expression may be modified by a clinician when assigning a treatment outcome to a patient in order to adaptively adjust a treatment outcome to particular patient needs. System 34 maintains an audit history in memory 32 of both modifications of a defined standard criteria treatment outcome description and of an associated logical expression accommodating patient specific criteria. This enables system 34 to generate user specified reports such as a report indicating how many patients are using standard treatment outcome requirements versus patient specific treatment outcome requirements in evaluating efficacy of a plan of care.

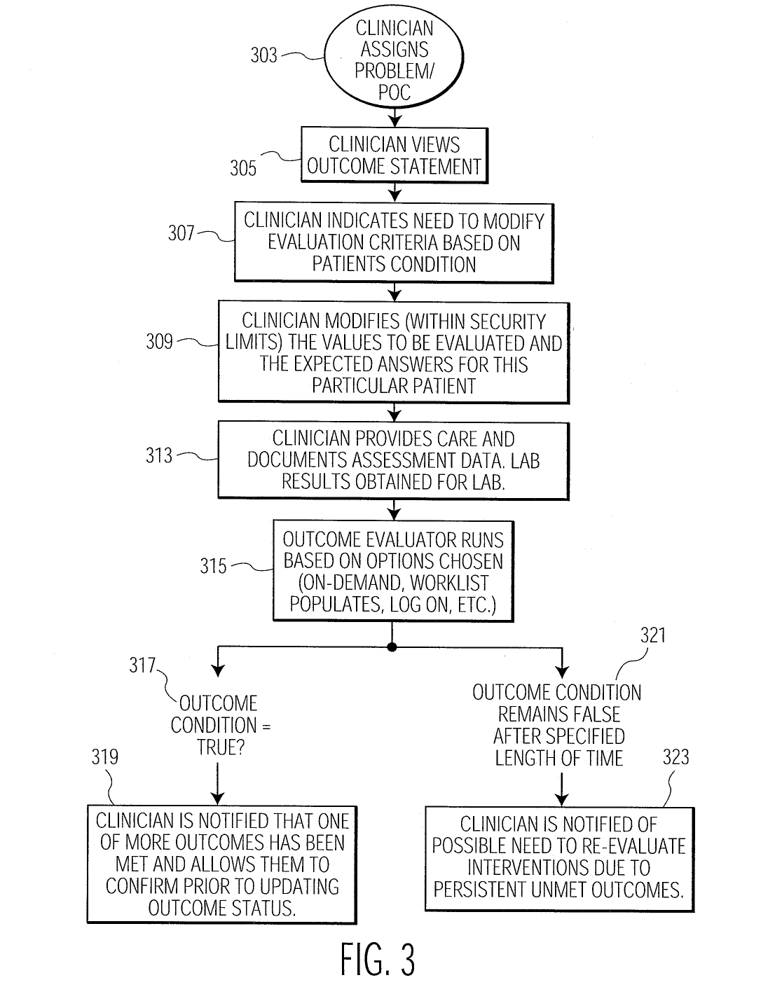

FIG. 3 shows a flowchart of a user managed process employing patient healthcare monitoring system 34 in making a patient assessment. In step 303 a clinician enters data, via user interface 40 in a plan of care, identifying a medical problem of a patient and in step 305 views an outcome statement. System 34 in step 307, provides a clinician, via user interface 40, with an associated treatment outcome description in text format derived from the Outcome catalog together with evaluation criteria (e.g., in response to a clinician command to view and/or modify evaluation criteria). The evaluation criteria comprise a logical expression to be evaluated to determine whether a treatment outcome has been met for treatment of the identified patient medical problem. In step 309 in response to a determination a clinician has necessary authorization, the clinician is provided with one or more display image menus via user interface 40 enabling the clinician to modify the patient parameter values to be evaluated as well as expected answers for the patient. The modification of the evaluation criteria includes modification of the logical expression to be evaluated to determine whether a treatment outcome has been met. The expression may be modified both in alteration of the patient parameters used in the expression and in the calculation performed by the logical expression.

In step 313 the clinician provides care and treatment to the patient in conformance with a care plan for the patient and using system 34 documents an assessment indicating progress of the patient for comparison with the care plan. The documented assessment includes visual and auditory Findings as well as laboratory test results of the patient, for example. In step 315, system 34 initiates performance of an evaluation of the patient using the previously generated evaluation criteria including a logical expression to be evaluated to determine whether a treatment outcome has been met for treatment of a patient medical condition. The logical expression is evaluated in response to a predetermined user selected event such as, upon user command, upon population of a healthcare worker worklist with outcomes overdue for evaluation and other outcomes, upon login by a clinician (evaluation is initiated for patients assigned to the clinician), upon storage of a patient assessment or upon a patient medical result being obtained and upon a care plan application being accessed. In step 317, if system 34 determines the logical expression evaluation provides a true result indicating a treatment outcome is met, the clinician is notified by system 34 in step 319, that one or more treatment outcomes for the patient are met. In addition system 34 further enables the clinician to review and confirm this result prior to updating outcome status of the patient in an electronic medical record of the patient. In step 321, if system 34 determines the logical expression evaluation provides a false result after expiration of a predetermined time duration, the clinician is notified by system 34 in step 323, of a possible need to review the treatment interventions, procedures and medications of the care plan in view of persistently unmet treatment outcomes required by a care plan.

Systems 34 and 24 (and other units in system 10) are implemented in software, but may also be implemented in hardware or a combination of both. The location of system 34 in server device 18 permits multiple systems and users to have access to monitoring system 34 from multiple client devices. Data storage unit 14 stores patient records, as well as other information for the hospital information system 10. Alternatively, the patient records may be stored in the database 38 in the memory unit 32 in the server device 18, in the memory unit 28 in the client device 12, or in memory units in the departmental systems 22. Patient records may also be stored in multiple decentralized memory units among the data storage unit 14, the client device 12, the server device 18, and the departmental systems 22. Patient records in the data storage unit 14 generally include any information related to a patient including, without limitation, biographical, financial, clinical, workflow, and care plan information.

The first local area network (LAN) 16 provides a communication network among the client device 12, the data storage unit 14 and the server device 18. The second local area network (LAN) 20 provides a communication network between the server device 18 and the departmental systems 22. The first LAN 16 and the second LAN 20 may be the same or different LANs, depending on the particular network configuration and the particular communication protocols implemented. Alternatively, one or both of the first LAN 16 and the second LAN 20 may be implemented as a wide area network (WAN).

The communication paths 52, 56, 60, 62, 64, 66, 68 and 70 permit the various elements, shown in FIG. 1, to communicate with the first LAN 16 or the second LAN 20. Each of the communication paths 52, 56, 60, 62, 64, 66, 68 and 70 are preferably adapted to use one or more data formats, otherwise called protocols, depending on the type and/or configuration of the various elements in the healthcare information systems 10. Examples of the information system data formats include, without limitation, an RS232 protocol, an Ethernet protocol, a Medical Interface Bus (MIB) compatible protocol, DICOM protocol, an Internet Protocol (I.P.) data format, a local area network (LAN) protocol, a wide area network (WAN) protocol, an IEEE bus compatible protocol, and a Health Level Seven (HL7) protocol. The communication paths 52, 56, 60, 62, 64, 66, 68 and 70 each may be formed as a wired or wireless (W/WL) connection.

The server device 18 generally includes a processor 30, a memory unit 32, and patient treatment monitoring system 34. The memory unit 32 includes workflow data and a database 38 containing patient records. Further, server device 18 may be implemented as a personal computer or a workstation. As previously mentioned, database 38 provides an alternate location for storing patient records, and user interface 23 is an alternate interface to interface 40 for a user. In the preferred embodiment of the present invention, monitoring system 34 is responsive to user interface 40 or user interface 23 in client device 12. In an alternative embodiment, monitoring system 24 is responsive to user interface 23 in client device 12.

Departmental systems 22 are systems that need access to information or provide information related to the health and/or welfare of patients in the care of the healthcare provider. Examples of the departmental systems 22 include, without limitation, a lab system 44, a pharmacy system 46, a financial system 48 and a nursing system 50, as shown in FIG. 1, but may also include a records system, a radiology system, an accounting system, a billing system, and any other system required or desired in a healthcare information system.

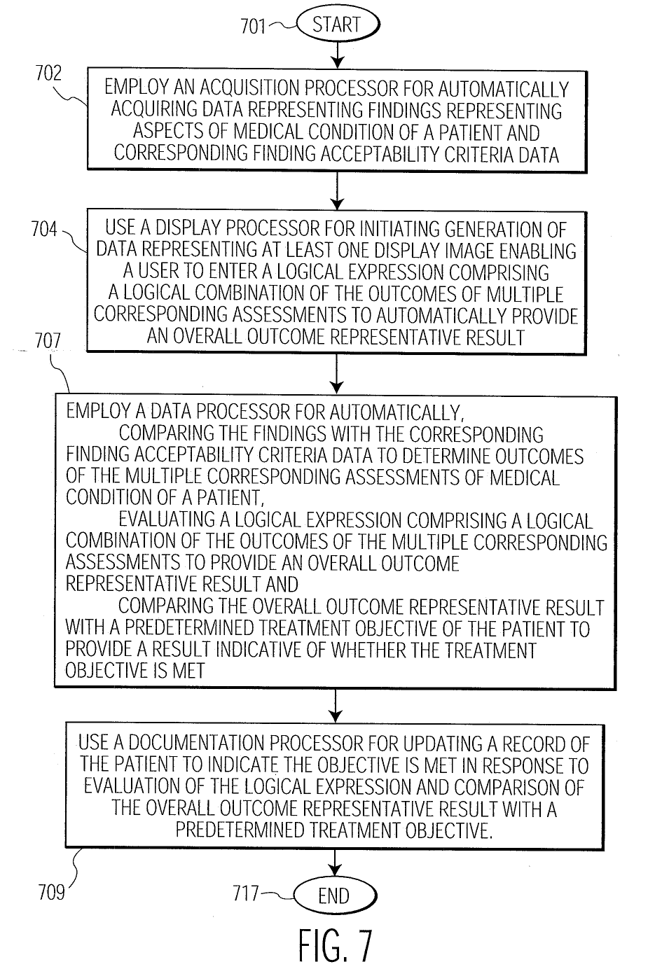

FIG. 7 shows a flowchart of a process used by patient healthcare monitoring system 34. In step 702 following the start at step 701, an acquisition processor in system 34 automatically acquires data representing Findings representing aspects of medical condition of a patient and corresponding Finding acceptability criteria data. The Findings are numeric, encoded or text and the Finding acceptability criteria are at least one of, (a) a maximum high value, (b) a minimum low value and (c) a range. The range comprises a normal range, an abnormal range or a critical range and the ranges are based on patient age and/or gender. A display processor in user interface 40 in step 704 initiates generation of data representing at least one display image enabling a user to enter a logical expression comprising a logical combination of the outcomes of multiple corresponding assessments to automatically provide an overall outcome representative result. An individual outcome of an assessment comprises a logical evaluation of a condition associated with a patient medical parameter. The logical evaluation of a condition associated with a patient medical parameter comprises a TRUE or FALSE determination or a text interpretation of user entered assessment text and employs at least one logical operator of, (a) less than, (b) less than or equal to, (c) greater than and (d) greater than or equal to. The logical combination employs at least one operator of, AND, OR, NOT, NOR, EXCLUSIVE OR and EXCLUSIVE NOR.

The logical combination comprises determining a logical AND of data representing the outcomes of multiple corresponding assessments to provide an overall outcome representative result. Data representing the outcomes of the multiple corresponding assessments individually comprise a logical 1 or 0 derived by an individual test to provide an overall outcome representative result. The logical 1 or 0 is derived in response to at least one of, (a) user data entry concerning a result of a patient assessment by a healthcare worker and (b) an automatically acquired test result provide by a medical parameter measurement or monitoring device attached to a patient.

A data processor in system 34 in step 707 automatically, compares the Findings with the corresponding Finding acceptability criteria data to determine outcomes of multiple corresponding assessments of medical condition of a patient, evaluates a logical expression comprising a logical combination of the outcomes of the multiple corresponding assessments to provide an overall outcome representative result and compares the overall outcome representative result with a predetermined treatment objective of the patient to provide a result indicative of whether the treatment objective is met.

A documentation processor in system 34 in step 709 updates a record of the patient to indicate the objective is met in response to evaluation of the logical expression and comparison of the overall outcome representative result with a predetermined treatment objective. The record of the patient comprises a care plan of the patient and the logical expression is associated with the care plan. The functions and steps performed by the acquisition processor, data processor and documentation processor of system 34 may be apportioned differently between these processors whereby some or all of the functions may be performed by one or more of these processors in any manner of distribution. The process of FIG. 7 terminates at step 717.

The systems presented in FIGS. 1-7 are not exclusive. Other systems and processes may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. A system according to invention principles is applicable to the health care industry and the real time management of patient outcomes is usable to alert care givers when patients are having adverse treatment outcomes. This allows for utilization of other processes within HIS 10 (using Workflow Engine and Rules Engine 42) to assist care givers in adjusting patient care to correct an adverse outcome. Further, any of the functions provided in the systems of FIGS. 1-7 may be implemented in hardware, software or a combination of both and may reside on one or more processing devices located at any location of a network linking the FIG. 1 elements or another linked network including another intra-net or the Internet.

What is claimed is:

1. A patient healthcare monitoring system, comprising:
an acquisition processor for automatically acquiring data representing outcomes of a plurality of corresponding assessments of medical condition of a patient;
a data processor for automatically,
evaluating a logical expression comprising a logical combination of said outcomes of said plurality of corresponding assessments to provide an overall outcome representative result and
comparing said overall outcome representative result with a predetermined treatment objective of said patient to provide a result indicative of whether said treatment objective is met; and
a documentation processor for updating a record of said patient to indicate said objective is met.

2. A system according to claim 1, wherein
said acquisition processor automatically acquires data representing Findings representing aspects of medical condition of a patient and corresponding Finding acceptability criteria data and for comparing said Findings with said corresponding Finding acceptability criteria data to determine said outcomes of said plurality of corresponding assessments of medical condition of said patient.

3. A system according to claim 1, wherein
said logical combination comprises determining a logical AND of data representing outcomes of said plurality of corresponding assessments to provide an overall outcome representative result.

4. A system according to claim 3, wherein
data representing outcomes of said plurality of corresponding assessments individually comprise a logical 1 or 0 derived by an individual test to provide an overall outcome representative result.

5. A system according to claim 4, wherein
said logical 1 or 0 is derived in response to at least one of, (a) user data entry concerning a result of a patient assessment by a healthcare worker and (b) an automatically acquired test result provide by a medical parameter measurement or monitoring device attached to a patient.

6. A system according to claim 1, wherein
said record of said patient comprises a care plan of said patient.

7. A system according to claim 6, wherein
said logical expression is associated with said care plan.

8. A user interface system for use in patient healthcare monitoring, comprising:
an acquisition processor for automatically acquiring data representing outcomes of a plurality of corresponding assessments of medical condition of a patient;
a display processor for initiating generation of data representing at least one display image enabling a user to enter a logical expression comprising a logical combination of said outcomes of said plurality of corresponding assessments to automatically provide an overall outcome representative result; and
a documentation processor for updating a record of said patient to indicate said objective is met in response to evaluation of said logical expression and comparison of said overall outcome representative result with a predetermined treatment objective.

9. A system according to claim 8, wherein
said acquisition processor automatically acquires data representing Findings representing aspects of medical condition of a patient and corresponding Finding acceptability criteria data, said Findings being for comparison with said corresponding Finding acceptability criteria data to determine said outcomes of said plurality of corresponding assessments of medical condition of said patient.

10. A system according to claim 8, wherein
said at least one display image enables a user to enter a logical expression comprising a logical combination of outcomes of said plurality of corresponding assessments and an individual outcome of an assessment comprises a logical evaluation of a condition associated with a patient medical parameter.

11. A system according to claim 10, wherein
said logical evaluation of a condition associated with a patient medical parameter comprises a TRUE or FALSE determination.

12. A system according to claim 10, wherein
said logical evaluation of a condition associated with a patient medical parameter comprises a text interpretation of user entered assessment text.

13. A system according to claim 10, wherein
said logical evaluation of a condition associated with a patient medical parameter employs at least one logical operator of, (a) less than, (b) less than or equal to, (c) greater than and (d) greater than or equal to.

14. A system according to claim 10, wherein
said logical combination employs at least one operator of, (a) AND, (b) OR, (c) NOT, (d) NOR, (e) EXCLUSIVE OR and (f) EXCLUSIVE NOR.

15. A patient healthcare monitoring system, comprising:
an acquisition processor for automatically acquiring data representing Findings representing aspects of medical condition of a patient and corresponding Finding acceptability criteria data;
a data processor for automatically,
comparing said Findings with said corresponding Finding acceptability criteria data to determine outcomes of a plurality of corresponding assessments of medical condition of a patient;
evaluating a logical expression comprising a logical combination of said outcomes of said plurality of corresponding assessments to provide an overall outcome representative result and
comparing said overall outcome representative result with a predetermined treatment objective of said patient to provide a result indicative of whether said treatment objective is met; and
a documentation processor for updating a record of said patient to indicate said objective is met.

16. A system according to claim 15, wherein
said Findings are at least one of, (a) numeric, (b) encoded and (c) text.

17. A system according to claim 15, wherein
said Finding acceptability criteria are at least one of, (a) a maximum high value, (b) a minimum low value and (c) a range.

18. A system according to claim 17, wherein
said range comprises at least one of, (a) a normal range, (h) an abnormal range and (c) a critical range and
said ranges are based on patient age and/or gender.
